(19) United States
(12) Patent Application Publication
(10) Pub. No.: US 2012/0225462 A1
(43) Pub. Date: Sep. 6, 2012

(54) NUTRIENT COMPOSITION FOR SACCHAROMYCES CEREVISIAE AND METHOD FOR USING THE SAME

(76) Inventors: Xuefeng YU, Yichang (CN); Zhihong LI, Yichang (CN); Minghua YU, Yichang (CN); Juan YAO, Yichang (CN); Jincheng LEI, Yichang (CN); Yinhu XU, Yichang (CN); Zhijun LI, Yichang (CN)

(21) Appl. No.: 13/469,063

(22) Filed: May 10, 2012

Related U.S. Application Data
(63) Continuation-in-part of application No. PCT/CN2011/002216, filed on Dec. 29, 2011.

(30) Foreign Application Priority Data
Jan. 10, 2011 (CN) 201110008032.2

(51) Int. Cl. C12P 7/04; C12N 9/50

(52) U.S. Cl. 435/157; 435/219

(57) ABSTRACT

Nutrient composition for saccharomyces cerevisiae and a method of using the same. The nutrient composition includes a yeast extract in 62-98 weight parts; an acid protease in 1.5-20 weight parts; and magnesium sulfate in 0.1-2.5 weight parts. The nutrient composition stimulates strong growth and reproduction of saccharomyces cerevisiae in various brewing materials, thereby speeding up alcoholic fermentation, shortening alcoholic fermentation period, and increasing liquor yield.

### CROSS-REFERENCE TO RELATED APPLICATIONS

This application is a continuation-in-part of International Patent Application No. PCT/CN2011/002216 with an international filing date of Dec. 29, 2011, designating the United States, now pending, and further claims priority benefits to Chinese Patent Application No. 201110008032.2 filed Jan. 10, 2011. The contents of all of the aforementioned applications, including any intervening amendments thereto, are incorporated herein by reference.

### CORRESPONDENCE ADDRESS

Inquiries from the public to applicants or assignees concerning this document should be directed to: MATTHIAS SCHOLL P. C., ATTN.: DR. MATTHIAS SCHOLL, ESQ., 14781 MEMORIAL DRIVE, SUITE 1319, HOUSTON, Tex. 77079.

### BACKGROUND OF THE INVENTION

1. Field of the Invention

The invention relates to a nutrient composition for saccharomyces cerevisiae and a usage method thereof.

2. Description of the Related Art

Nutrients are essential for the life activities of saccharomyces cerevisiae, without it, saccharomyces cerevisiae will come to an end. Nutritional deficiency or insufficient nutrients would cause saccharomyces cerevisiae to grow slowly or less strong, eventually leading to slow and incomplete alcoholic fermentation and low liquor yield. In actual production of alcohol and brewing, although raw materials contain rich nutrients, there still have some nutrients having insufficient content, such as nitrogen, magnesium, and phosphorus. The brewing materials also lacks some vitamins essential for the strong growth of the yeast, for example, the content of biotin is very low in molasses, therefore, extra biotin has to be added.

Major nutrient sources for saccharomyces cerevisiae include a carbon source, a nitrogen source, inorganic salts, and vitamins.

The carbon source is rich in raw materials and thus does not need supplementing.

The nitrogen source constitutes proteins and nucleases in a yeast cell and serves as a nitrogen source for catabolites. The inorganic salts are indispensable substances for life activities of yeasts with main functions of: being a component of a cell; being an integral part of an enzyme reactive group or maintaining activities of an enzyme; adjusting osmotic pressure, pH, oxidation-reduction potential, etc; and being an energy source for an autotroph.

Main inorganic salts in a yeast cell include magnesium salts and phosphates, in which the magnesium salts often function as an activator for most of the enzymatic reactions. In a yeast, the content of magnesium salts (MgO) accounts for approx. 0.45% of its dry weight and 3.77-6.34% of the yeast ash content. Under normal circumstances, effective MgO content is insuifficient in raw materials, and thus it needs to be supplemented. Phosphates account for approx. half (44.8%-59.4%) of the yeast ash content. Phosphorus is a component element of nucleic acid, phosphatide, and many coenzymes, such as coenzyme 1, coenzyme 2, and coenzyme A. It involves in the phosphorylation process of carbohydrates to produce energy-rich phosphate (ATP) for energy storage and tranmission. Meanwhile, the phosphates are important pH buffers.

Different types of vitamins are required for the growth of yeasts including biotin, pantothenic acid, inositol, and thiamine. Generally, except those mentioned above, other types of vitamins can be provided in raw materials for the growth of the yeasts.

Until now, there has not been a solution in the art to combine a yeast extract, acid protease, and magnesium sulfate in a reasonable proportion to develop a nutrient composition for saccharomyces cerevisiae with significant effects.

### SUMMARY OF THE INVENTION

In view of the above-described problems, it is one objective of the invention to provide a highly effective nutrient composition for saccharomyces cerevisiae comprising a yeast extract, acid protease, and magnesium sulfate according to a scientific proportion. It solves the disadvantages in actual production of alcohol and brewing, for example, slow or incomplete alcoholic fermentation and low liquor yield caused by insufficient nutrients in raw materials for the growth of yeasts and manual supplementation of nutrients, which is single and incomplete, or the addition amount is either too much or too little. The nutrient composition for saccharomyces cerevisiae provided by the invention can reduce the fermentation period by 5-15 hrs and increase alcohol by volume (ABV) by 1-2% (v/v).

In accordance with one embodiment of the invention, there provided is a nutrient composition for saccharomyces cerevisiae comprising:
a yeast extract: 62-98 weight parts;
an acid protease: 1.5-20 weight parts; and
magnesium sulfate: 0.1-2.5 weight parts.

In a class of this embodiment, the nutrient composition comprises:
the yeast extract: 85-95 weight parts;
the acid protease: 4-13 weight parts; and
magnesium sulfate: 0.5-1.5 weight parts.

In a class of this embodiment, the nutrient composition comprises:
the yeast extract: 90 weight parts;
the acid protease: 9 weight parts; and
magnesium sulfate: 1.0 weight part.

Amount of the acid protease used in the invention is calculated based on enzyme activity unit of 50,000 u/g. If the activity unit thereof is not 50,000 u/g, the amount of the acid protease must be changed accordingly.

In a class of this embodiment, the yeast extract is produced using food yeasts with rich proteins as the raw material and by modern biotechnology, such as autolysis and refinement. It is rich in nitrogen, phosphorus, amino acid, biotin, pantothenic acid, inositol, and thiamine. Yeast extract, such as yeast extract powder FM818, yeast autolytic powder FM901, and FM801, can be bought from Angel Yeast Co., Ltd.

In a class of this embodiment, the acid protease is a type of solid and gray powdered enzyme with activity at approx. 50,000 u/g and it is easily accessible from companies producing enzyme preparation. In the invention, different types of acid proteases can be used.

In a class of this embodiment, magnesium sulfate is a type of solid and white chemical material with fine granules and it is easily accessible from companies selling chemical materials.

In a class of this embodiment, the nutrient composition further comprises zinc sulfate, ammonium dibasic phosphate, and calcium chloride, which make the nutrient composition for saccharomyces cerevisiae have more complete nutrients, more stable performance, and wider applicable materials.

The nutrient composition provided by the invention are applicable to the alcoholic fermentation of not only starch materials, such as corn, cassava, wheat, and rice, but also saccharine materials, such as molasses, sucrose, and sugar sorghum.

The nutrient composition provided by the invention is added according to a certain proportion during the fermentation process. The addition proportion is a ratio of the mass of the nutrient composition added to the mass of raw materials to be fermented. Generally such a proportion is 0.1-1% (w/w), perferably 0.4-0.6% (w/w), and more preferably 0.5% (w/w).

The nutrient composition provided by the invention can stimulate strong growth and reproduction of saccharomyces cerevisiae in various brewing materials, thereby speeding up alcoholic fermentation, shortening alcoholic fermentation period, and increasing liquor yield. The nutrient composition can shorten the alcoholic fermentation period by 5-15 hrs thereby enhancing the equipment utilization, and increase final ABV by 1-2% (v-v) thereby saving the production costs of alcohol per ton by RMB 50-200.

### DETAILED DESCRIPTION OF THE EMBODIMENTS

For further illustrating the invention, experiments detailing a nutrient composition for saccharomyces cerevisiae and a usage method thereof are described below. It should be noted that the following examples are intended to describe and not to limit the invention.

All raw materials and instruments used in the examples are shown in table 1:

TABLE 1

| Raw Materials | Origin/Manufacturers | Model |
|---|---|---|
| Thermostable amylase | Novozymes (China) | 20,000 u/mL |
| | Investment Co Ltd. | |
| Glucoamylase | Novozymes (China) | 100,000 u/mL |
| | Investment Co Ltd. | |
| Thermostable | Angel Yeast Co., Ltd. | Dry yeast with |
| saccharomyces | | high-temperature |
| cerevisiae | | resistance and high |
| | | activity from |
| | | Angel Yeast Co., |
| | | Ltd. |
| Alcoholmeter | Qingxian Measuring | 10-20% vol |
| | Glass Apparatus | |
| | Factory | |

Corn is used as a raw material in the following embodiments for brewing using conventional fermentation methods. In the process of brewing, a nutrient is added to evaluate its influence on the alcoholic fermentation process.

Fermentation Procedures

1. Selection of raw materials: sieve the corn powder with a 1.5 mm mesh sieve.

2. Moisture treatment: add 240 g of water with a temperature of 60-70° C. into 100 g of the corn powder obtained from the step 1 based on a material-water ratio of 1:2.4 and moistens the material for 30 mins.

3. Liquefaction: add a thermostable amylase for every 10 u/g corn powder (or 10-20 u/g corn powder) and liquefy them under 96° C. (95-97° C.) for 1.5 hrs (1.5-2 hrs), afterwards, cool them down to 32° C. and adjust pH=4.2 (pH 4.2-4.5) with 50% dilute sulfuric acid.

4. Add a nutrient for saccharomyces cerevisiae of the invention.

5. Liquor fermentation: add glucoamylase for every 180 u/g (150-200 u/g) corn powder and dry yeast with high-temperature resistance and high activity from Angel Yeast Co., Ltd. based on 0.2% (w/w) corn powder, and then ferment under 33° C. for 72 hrs.

6. Measure the weight loss of carbon dioxide once per 24 hrs.

7. Distillation: make liquor by distillation with conventional brewing processes i.e. measure 100 mL fermented liquor using a graduated cylinder and put it into a 1000 mL distillation flask and then add 1-2 drops of anti-foaming agent (e.g. salad oil) for fermentation. Collect the distillates using a 100 mL measuring flask (with an ice water bath). When the distillates reach approx. 95 mL, stop distillation and take the measuring flask down and when the temperature reaches the room temperature, dilute the solution to 100 mL.

8. Measurement of ABV: put the diluted distillates into a clean and dry graduated cylinder (100 mL), stand for a few minutes until air bubbles disappear from the liquor and then transfer to a clean and precise alcoholmeter and slightly press once. Stand for a while, observe the scale value at the tangent point of the lowest arc surface of the liquid at eye level and measure the temperature of the liquid, convert it to the ABV under 20° C. according to Alcoholmeter Temperature and Concentration Conversion Table.

### EXAMPLE 1

### Influence of Yeast Extract on Alcoholic Fermentation of Corn

The above-mentioned fermentation procedures were carried out for alcoholic fermentation of corn, and the nutrient added in step 4 was a yeast extract (yeast extract powder FM818 produced and provided by Angel Yeast Co., Ltd.) with amount (based on weight percentage of fermentation material) of 0, 0.05%, 0.1%, 0.25%, and 0.50%, respectively. The chemical properties of the yeast extract are listed in table 2.

TABLE 2

| Appearance | Yellow or brown, powdery | |
|---|---|---|
| Smell | Smell of yeast extract | |
| Total nitrogen (dry weight) | ≧10.0% | |
| Ammonia nitrogen (dry weight) | ≧4.0% | |
| Moisture | ≦6.0% | |
| Sodium chloride | ≦2.0% | |
| Ash | ≦15.0% | |
| pH (2% aqueous solution) | 5.3-7.2 | |
| Lead | ≦2.0 | mg/kg |
| Total arsenic | ≦2.0 | mg/kg |
| Total number of colonies | ≦5 × 10⁴ | cfu/g |
| Coliform | ≦0.3 | MPN/g |

| | |
|---|---|
| Salmonella | None |
| Staphylococcus aureus | None |
| Shigella | None |

The weight loss of carbon dioxide (g) was measured every 24 hrs. When the reaction was over, the final ABV (v/v) was measured. The test results are shown in table 3.

TABLE 3
Influence of Yeast Extract on Alcoholic Fermentation of Corn

| Yeast extract (%) | 0.00 | 0.05 | 0.1 | 0.25 | 0.50 |
|---|---|---|---|---|---|
| 0-24 hrs weight loss | 11.94 | 11.72 | 11.37 | 13.35 | 19.07 |
| of carbon dioxide (g) | | | | | |
| 24-48 hrs weight loss | 12.40 | 11.98 | 12.65 | 11.66 | 12.39 |
| of carbon dioxide (g) | | | | | |
| 48-72 hrs weight loss | 9.73 | 10.73 | 10.69 | 9.78 | 3.89 |
| of carbon dioxide (g) | | | | | |
| Total weight loss of | 34.07 | 34.43 | 34.71 | 34.79 | 35.35 |
| carbon dioxide (g) | | | | | |
| ABV (v/v) | 12.90 | 13.20 | 13.40 | 13.40 | 13.70 |

As shown in table 3, the yeast extract has a big influence on alcoholic fermentation of corn, the ABV is increased, and based on weight loss of carbon dioxide, and the fermentation speed is also enhanced significantly.

### EXAMPLE 2

### Influence of Acid Protease on Alcoholic Fermentation of Corn

The above-mentioned fermentation procedures were carried out for alcoholic fermentation of corn, and the nutrient added in step 4 was an acid protease (provided by Novozymes (China) Investment Co Ltd.; enzyme activity 50,000 u/g) with amount (based on weight percentage of fermentation material) of 0, 0.01%, 0.03%, 0.05%, and 0.07%, respectively. The weight loss of carbon dioxide (g) was measured every 24 hrs. When the reaction was over, the final ABV (v/v) was measured. The test results are shown in table 4.

TABLE 4

| Acid protease (%) | 0.00 | 0.01 | 0.03 | 0.05 | 0.07 |
|---|---|---|---|---|---|
| 0-24 hrs weight loss | 11.85 | 20.10 | 21.85 | 19.86 | 15.07 |
| of carbon dioxide (g) | | | | | |
| 24-48 hrs weight loss | 12.78 | 12.42 | 12.13 | 13.75 | 19.71 |
| of carbon dioxide (g) | | | | | |
| 48-72 hrs weight loss | 9.38 | 2.12 | 1.37 | 1.86 | 0.29 |
| of carbon dioxide (g) | | | | | |
| Total weight loss of | 34.01 | 34.64 | 35.35 | 35.47 | 35.07 |
| carbon dioxide (g) | | | | | |
| ABV | 12.90 | 13.50 | 13.80 | 13.80 | 13.70 |

As shown in table 4, the acid protease has a big influence on alcoholic fermentation of corn, the ABV is increased accordingly, and based on weight loss of carbon dioxide, and the fermentation speed is also enhanced significantly.

### EXAMPLE 3

### Influence of Magnesium Sulfate on Alcoholic Fermentation of Corn

The above-mentioned fermentation procedures were carried out for alcoholic fermentation of corn, and the nutrient added in step 4 was magnesium sulfate (provided by Yichang Xilong Chemical Store) with amount (based on weight percentage of fermentation material) of 0, 0.001%, 0.003%, 0.005%, and 0.007%, respectively. The weight loss of carbon dioxide (g) was measured every 24 hrs. When the reaction was over, the final ABV (v/v) was measured. The test results are shown in table 5.

TABLE 5

| Magnesium sulfate (%) | 0 | 0.001 | 0.003 | 0.005 | 0.007 |
|---|---|---|---|---|---|
| 0-24 hrs weight loss | 11.83 | 12.96 | 13.06 | 12.85 | 12.23 |
| of carbon dioxide (g) | | | | | |
| 24-48 hrs weight loss | 13.64 | 18.81 | 18.37 | 18.46 | 18.89 |
| of carbon dioxide (g) | | | | | |
| 48-72 hrs weight loss | 8.58 | 3.11 | 3.39 | 3.51 | 3.69 |
| of carbon dioxide (g) | | | | | |
| Total weight loss of | 34.05 | 34.88 | 34.82 | 34.82 | 34.81 |
| carbon dioxide (g) | | | | | |
| ABV (v/v) | 12.90 | 13.6 | 13.5 | 13.6 | 13.6 |

As shown in table 5, the magnesium sulfate has a big influence on alcoholic fermentation of corn, the ABV is increased, and based on weight loss of carbon dioxide, and the fermentation speed is also enhanced significantly.

### EXAMPLE 4

### Influence of Yeast Extract, Acid Protease, and Magnesium Sulfate on Alcoholic Fermentation of Corn

The above-mentioned fermentation procedures were carried out for alcoholic fermentation of corn, and the nutrient added in step 4 was a yeast extract, acid protease, magnesium sulfate, or a mixture thereof, with amount of 0.5%. The weight loss of carbon dioxide (g) was measured every 24 hrs. When the reaction was over, the final ABV (v/v) was measured. The test results are shown in table 6.

TABLE 6

| | | | Acid | Magne- | Yeast Extract + |
|---|---|---|---|---|---|
| | | Yeast | Pro- | sium | Acid |
| | Blank | Extract | tease | Sulfate | Protease (1:1) |
| Amount added (%) | 0 | 0.5 | 0.5 | 0.5 | 0.5 |
| 0-24 hrs weight loss | 12.78 | 19.11 | 20.20 | 13.08 | 20.75 |
| of carbon dioxide (g) | | | | | |
| 24-48 hrs weight loss | 11.85 | 12.19 | 13.19 | 18.81 | 11.37 |
| of carbon dioxide (g) | | | | | |
| 48-72 hrs weight loss | 9.47 | 3.91 | 2.03 | 3.03 | 3.10 |
| of carbon dioxide (g) | | | | | |
| Total weight loss of | 34.10 | 35.21 | 35.42 | 34.92 | 35.22 |
| carbon dioxide (g) | | | | | |
| ABV (v/v) | 12.9 | 13.6 | 13.8 | 13.5 | 13.8 |

| | Yeast | Acid | Yeast Extract + |
|---|---|---|---|
| | Extract + | Protease + | Acid Protease + |
| | Magnesium | Magnesium | Magnesium |
| | Sulfate (1:1) | Sulfate (1:1) | Sulfate (1:1:1) |
| Amount added (%) | 0.5 | 0.5 | 0.5 |
| 0-24 hrs weight loss | 19.35 | 20.75 | 22.35 |
| of carbon dioxide (g) | | | |
| 24-48 hrs weight loss | 11.75 | 12.35 | 13.18 |
| of carbon dioxide (g) | | | |
| 48-72 hrs weight loss | 4.02 | 2.11 | 0.51 |
| of carbon dioxide (g) | | | |
| Total weight loss of | 35.12 | 35.21 | 36.04 |
| carbon dioxide (g) | | | |
| ABV (v/v) | 13.7 | 13.8 | 14.0 |

As shown in table 6, the combination of the yeast extract, acid protease, and magnesium sulfate the magnesium sulfate has bigger influence on alcoholic fermentation of corn. The fermentation speed and ABV are increased significantly. The combination of those three substances has much more noticeable effect than that produced by an individual one.

### EXAMPLE 5

### Influence of Nutrient Composition for Saccharomyces Cerevisiae on Alcoholic Fermentation of Corn

The above-mentioned fermentation procedures were carried out for alcoholic fermentation of corn, and the nutrient added in step 4 was listed in table 7. The percentage of each nutrient to be added and the test results are shown in table 7.

TABLE 7

| | | | Test | Test | Test | Test | Test | Test |
|---|---|---|---|---|---|---|---|---|
| | | Blank | 1 | 2 | 3 | 4 | 5 | 6 |
| Formula | Yeast | 0 | 90 | 98 | 95 | 85 | 70 | 62 |
| | Extract | | | | | | | |
| | Acid | 0 | 9 | 1.5 | 4 | 20 | 13 | 3 |
| | Protease | | | | | | | |
| | Magnesium | 0 | 1.0 | 0.5 | 1.5 | 0.1 | 2.5 | 2.0 |
| | Sulfate | | | | | | | |

| | | | | | | | |
|---|---|---|---|---|---|---|---|
| 0-12 hrs weight loss | 5.1 | 10.07 | 9.22 | 10.04 | 10.02 | 9.89 | 9.55 |
| of carbon dioxide (g) | | | | | | | |
| 12-24 hrs weight loss | 6.65 | 12.38 | 11.45 | 11.93 | 11.62 | 11.60 | 11.53 |
| of carbon dioxide (g) | | | | | | | |
| 24-36 hrs weight loss | 6.85 | 9.05 | 8.62 | 8.80 | 8.95 | 8.97 | 8.58 |
| of carbon dioxide (g) | | | | | | | |
| 36-48 hrs weight loss | 6.4 | 4.82 | 5.96 | 4.92 | 4.96 | 4.93 | 5.69 |
| of carbon dioxide (g) | | | | | | | |
| 48-64 hrs weight loss | 5.72 | 0.26 | 1.02 | 0.79 | 0.86 | 0.82 | 0.88 |
| of carbon dioxide (g) | | | | | | | |
| 64-72 hrs weight loss | 3.3 | 0 | 0 | 0 | 0 | 0 | 0 |
| of carbon dioxide (g) | | | | | | | |
| Total weight loss of | 34.02 | 36.58 | 36.27 | 36.48 | 36.41 | 36.21 | 36.23 |
| carbon dioxide (g) | | | | | | | |
| ABV (v/v) | 12.8 | 14.5 | 14.2 | 14.4 | 14.3 | 14.2 | 14.2 |

As shown in table 7, the nutrient composition for saccharomyces cerevisiae has significant influence on the alcoholic fermentation of corn. The fermentation speed is increased significantly, fermentation period is reduced by more than 12 hours, ABV is increased by 1.7 (v/v), and the liquor yield (ABV) is largely increased. The above tests show that the preferred percentage of the nutrient composition for saccharomyces cerevisiae by weight is that yeast extract 90, acid protease 9, and magnesium sulfate 1.0.

### EXAMPLE 6

### Influence of Added Proportion of Nutrient Composition for Saccharomyces Cerevisiae on Alcoholic Fermentation of Corn

The above-mentioned fermentation procedures were carried out for alcoholic fermentation of corn, and the nutrient added in step 4 was the nutrient composition for saccharomyces cerevisiaes with the proportion listed in test 1 of table 7. The addition proportion of the nutrient composition for saccharomyces cerevisiaes, which was a weight percent of the fermentation material, is listed in table 8. When the reaction was over, final ABV (v/v) was measured. The test results are shown in table 8.

TABLE 8

| | Nutrient composition for saccharomyces cerevisiae (%) | | | | | | | | | | |
|---|---|---|---|---|---|---|---|---|---|---|---|
| | 0 | 0.1 | 0.2 | 0.3 | 0.4 | 0.5 | 0.6 | 0.7 | 0.8 | 0.9 | 1 |
| ABV (v/v) | 12.8 | 13.9 | 13.9 | 14.2 | 14.4 | 14.5 | 14.5 | 14.5 | 14.5 | 14.5 | 14.5 |

As shown in table 8, the ABV is increased significantly when the nutrient composition for saccharomyces cerevisiaes are added with the amount of 0.1%, 0.2%, 0.3%, 0.4%, 0.5%, 0.6%, 0.7%, 0.8%, 0.9%, and 1% of the fermentation material by weight. In terms of fermentation results and use costs, 0.4-0.6% of addition proportion is more appropriate and 0.5% (based on weight ratio of fermentation material) is more preferred.

### EXAMPLE 7

### Influence of Nutrient Composition for Saccharomyces Cerevisiae Added with Cofactor (e.g. Zinc Sulfate) on Alcoholic Fermentation of Corn

The above-mentioned fermentation procedures were carried out for alcoholic fermentation of corn, and the nutrient added in step 4 was the nutrient composition for saccharomyces cerevisiaes with the proportion listed in test 1 of table 7, or zinc sulfate was further added to the nutrients listed in test 1 of table 7 accounting for 0.5% of the total amount. Each of the above two types of nutrients accounts for 0.5% of the added fermentation material. When the reaction was over, the final ABV (v/v) was measured. The test results are shown in table 9.

TABLE 9

| | | Nutrient composition for | Nutrient composition for |
|---|---|---|---|
| | | saccharomyces cerevisiae | saccharomyces cerevisiae |
| | Blank | (incl. 0.5% zinc sulfate) | (excl. zinc sulfate) |
| ABV | 12.8 | 14.6 | 14.5 |
| (v/v) | | | |

The fermentation results show that the ABV is increased by 0.1 (v/v) in alcoholic fermentation of corn with the nutrient composition added with the cofactor zinc sulfate compared with the one without the zinc sulfate.

The main purpose of adding a cofactor is to make the nutrient composition for saccharomyces cerevisiae have complete nutrients, stable performance, and wide applicable materials. It is applicable to the alcoholic fermentation of not only starch materials, such as corn, cassava, wheat, and rice, but also saccharine materials, such as molasses, sucrose, and sugar sorghum.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A nutrient composition for saccharomyces cerevisiae, comprising:
a) a yeast extract: 62-98 weight parts;
b) an acid protease: 1.5-20 weight parts; and
c) magnesium sulfate: 0.1-2.5 weight parts.

2. The nutrient composition of claim 1, comprising:
a) the yeast extract: 89-95 weight parts;
b) the acid protease: 4-13 weight parts; and
c) magnesium sulfate: 0.5-1.5 weight parts.

3. The nutrient composition of claim 2, comprising:
a) the yeast extract: 90 weight parts;
b) the acid protease: 9 weight parts; and
c) magnesium sulfate: 1.0 weight part.

4. The nutrient composition of claim 1, further comprising zinc sulfate, ammonium dibasic phosphate, calcium chloride, or a mixture thereof.

5. The nutrient composition of claim 3, further comprising zinc sulfate, ammonium dibasic phosphate, calcium chloride, or a mixture thereof.

6. A method of fermentation comprising applying the nutrient composition of claim 1, a usage percentage of the nutrient composition being 0.1-1 wt. % of a fermentation material.

7. The method of claim 6, wherein the usage percentage of the nutrient composition is 0.4-0.6 wt. % of the fermentation material.

8. The method of claim 7, wherein the usage percentage of the nutrient composition being 0.5 wt. % of the fermentation material.

9. The method of claim 6, wherein the fermentation material is a starch material or saccharine material.

10. The method of claim 8, wherein the fermentation material is a starch material or saccharine material.

11. The method of claim 6, wherein the nutrient composition further comprising zinc sulfate, ammonium dibasic phosphate, calcium chloride, or a mixture thereof.

12. The method of claim 10, wherein the nutrient composition further comprising zinc sulfate, ammonium dibasic phosphate, calcium chloride, or a mixture thereof.
